(19) United States
(12) Patent Application Publication
(10) Pub. No.: US 2025/0186723 A1
(43) Pub. Date: Jun. 12, 2025

(54) CONTINUOUS POSITIVE AIRWAY PRESSURE CPAP DEVICE FEATURED WITH ACTIVE NOISE CANCELLATION AND METHOD THEREOF

(71) Applicant: KONINKLIJKE PHILIPS N.V., EINDHOVEN (NL)

(72) Inventor: BERNARD F. HETE, KITTANNING, PA (US)

(21) Appl. No.: 18/953,446

(22) Filed: Nov. 20, 2024

Related U.S. Application Data
(60) Provisional application No. 63/607,594, filed on Dec. 8, 2023.

(51) Int. Cl. A61M 16/00; A61M 16/06; A61M 16/08

(52) U.S. Cl. CPC A61M 16/024; A61M 16/0003; A61M 16/06; A61M 16/0883; A61M 2205/3344; A61M 2205/3375; A61M 2205/42; A61M 2205/50; A61M 2209/088; A61M 2210/0618; A61M 2210/0662

(57) ABSTRACT

A continuous positive airway pressure CPAP device featured with active noise cancellation comprises at least a patient interface device, a microphone, a controller, and an audio output device. The patient interface device is fluidly coupled to a base station via a patient circuit for delivery of a pressurized flow of breathable gas. The microphone is acoustically coupled to the CPAP device for capturing regular or random noise generated by or at the CPAP device at least during the delivery of breathable gas. The controller processes the captured noise, via an active noise cancellation ANC algorithm, and generates an ANC output signal. The audio output device, coupled to the patient interface device, generates a CPAP device noise cancellation audio output based on the ANC output signal, wherein the audio output reduces noise effects by eliminating up to a given noise threshold amount of the CPAP device noise.

### CROSS REFERENCE TO RELATED APPLICATION

This application claims the benefit of U.S. Provisional Patent Application No. 63/607,594 filed Dec. 8, 2023. This application is incorporated by reference herein.

### BACKGROUND

The present embodiments relate generally to positive airway pressure devices and more particularly, to a continuous positive airway pressure (CPAP) device featured with active noise cancellation (ANC) and a method thereof.

Continuous positive air pressure (CPAP) devices are known to produce noise during use. For example, noise produced during use includes noise emanating from a blower or air flow generator that provides a pressurized flow of breathable gas. Such noise produced during usage is undesired by the device user and may cause the user to avoid using the CPAP device according to a treatment plan because of the undesired noise. Accordingly, it would be desirable to solve the problem associated with noise from CPAP devices and render such devices quieter during operation or use.

Active noise cancellation (ANC) has been considered as a solution, but it is well-known that ANC does not work in large open systems like a CPAP emanating noise into open three-dimensional (3D) space. That is, ANC does not work well when applied directly to multi-point noise sources emanating into open 3D space.

Accordingly, an improved method and apparatus for overcoming the problems in the art is desired.

### SUMMARY

According to one aspect, the embodiments of the present disclosure take advantage of the fact that ANC is known to not work in large open systems that emanate noise into open three-dimensional (3D) space. However, the embodiments advantageously use ANC which does work well when applied to the ear of a person or receiver as closely as possible.

According to one embodiment, a continuous positive airway pressure CPAP device featured with active noise cancellation comprises at least a patient interface device, a microphone, a controller, and an audio output device. The patient interface device is configured for being fluidly coupled to a base station of the CPAP device via a patient circuit for delivery of a pressurized flow of breathable gas from the CPAP device to the patient interface device, whereby a subject wearing the patient interface device would receive a corresponding treatment of the pressurized flow of breathable gas. The microphone is adapted for being acoustically coupled to the CPAP device for capturing regular or random noise generated by or at the CPAP device at least during the delivery of the pressurized flow of breathable gas to the subject. The controller is adapted to implement an active noise cancellation ANC algorithm for (i) processing the captured regular or random noise and (ii) generating an ANC output signal based on the processed regular or random noise. The audio output device is adapted for being coupled to the patient interface device at a predetermined placement location of the patient interface device, e.g., proximate one or both ears of the subject, at least during the delivery of the pressurized flow of breathable gas. The audio output device receives the ANC output signal and generates, in response to receipt of the ANC output signal, a CPAP device noise cancellation audio output. The CPAP device noise cancellation audio output is configured to reduce noise effects of the CPAP device by eliminating up to a given noise threshold amount of the regular or random noise generated by or at the CPAP device.

According to one embodiment, the predetermined placement location of the audio output device comprises a location of one or more components of the audio output device placed within one or both ears of a subject. According to another embodiment, the predetermined placement location of the audio output device comprises a location of one or more component of the audio output device placed overlying both ears of a subject. According to yet another embodiment, the placement location of the audio output device comprises a location of a component of the audio output device placed overlying at least one ear of a subject. According to a still further embodiment, the placement location of the audio output device comprises a location of the audio output device placed within a given threshold distance from at least one ear of a subject.

According to another embodiment, the given noise threshold amount can comprise at least one of (i) more than 50%, (ii) more than 75%, and (iii) more than 90% of the regular or random noise generated by the CPAP device. For example, regular CPAP device noise may consist of noise having a given duty cycle or periodic characteristic behaviour, whereas random CPAP device noise may consist of noise other than regular CPAP device noise. In one embodiment, the regular and/or random CPAP device noise may consist of more than 90% of noise which emanates from a blower alone in the base station of the CPAP device. In another embodiment, the audio output device comprises one or more selected from the group consisting of (i) ear buds, (ii) headphones, (iii) a flexible audio output disk (e.g., for placement during use on a pillow or integrated into the pillow), and (iv) a flexible audio output material (e.g., integrated into a CPAP mask, headgear, or other patient interface device).

According to yet another embodiment, the patient interface device includes a CPAP headgear component, and wherein the audio output device is either (i) physically attached to the CPAP headgear component or (ii) physically separate from the CPAP headgear component. In another embodiment, the patient interface device comprises one selected from the group consisting of a nasal mask, a full facial mask, and a CPAP patient interface device configuration other than the nasal mask and full facial mask. For example, the CPAP patient interface device configuration may include a nasal pillow mask.

In yet another embodiment, the microphone is coupled inside a housing of the base station of the CPAP device for receiving the regular or random noise generated by the CPAP device at least during the delivery of the pressurized flow of breathable gas. For example, the microphone is preferably located inside the base station housing, adjacent to or within a predetermined limited distance from, but not inside, the blower or blower housing which provides the flow of breathable gas. In other words, the predetermined distance can comprise a distance which maximizes a noise signal strength of the regular or random noise emanating from the blower and/or blower housing during delivery of the pressurized flow of breathable gas. Such a predetermined proximal distance is distinguishable from distal distances, e.g., in comparison to a location remote from the blower or blower housing within the base station housing which may contribute to a diminished noise signal strength.

According to another embodiment, a method of active noise cancellation with a continuous positive airway pressure CPAP device, comprising the following steps. A first step includes delivering a pressurized flow of breathable gas to a subject, via a patient interface device fluidly coupled to a base station of the CPAP device via a patient circuit. During at least the delivery of the pressurized flow of breathable gas, the CPAP device produces regular and/or random CPAP device noise. For example, the regular CPAP device noise can consist of noise having a given duty cycle or periodic characteristic behaviour, whereas random CPAP device noise may consist of noise other than regular CPAP device noise. In one embodiment, the regular and/or random CPAP device noise may consist of more than 90% of noise which emanates from a blower alone in the base station of the CPAP device.

A second step includes recording, via a microphone acoustically coupled to the CPAP device, the CPAP device noise produced by the CPAP device at least during the delivery of the pressurized flow of breathable gas to the subject. A third step includes implementing, via a controller, an active noise cancellation (ANC) algorithm for (i) processing the recorded CPAP device noise and (ii) generating a CPAP anti-noise wave signal based on the processed CPAP device noise. A subsequent step includes outputting, via an audio output device coupled to the patient interface device at a predetermined placement location of the patient interface device (e.g., proximate one or both ears of the subject) at least during the delivery of the pressurized flow of breathable gas, a CPAP device noise cancellation audio output signal based on the CPAP anti-noise wave signal. The CPAP device noise cancellation audio output signal is configured to reduce noise effects of the CPAP device by eliminating up to a given noise threshold amount of the regular and/or random noise produced by or at the CPAP device.

According to one embodiment, the method may include wherein the predetermined placement location of the audio output device comprises a location of one or more component of the audio output device placed within one or both ears of a subject. According to another embodiment, the method may include wherein the predetermined placement location of the audio output device comprises a location of one or more component of the audio output device placed overlying both ears of a subject. According to yet another embodiment, the method may include wherein the placement location of the audio output device comprises a location of a component of the audio output device placed overlying at least one ear of a subject. According to a still further embodiment, the placement location of the audio output device may comprise a location of the audio output device placed within a given threshold distance from at least one ear of a subject.

According to another embodiment, the method includes wherein the given noise threshold amount can comprise at least one of (i) more than 50%, (ii) more than 75%, and (iii) more than 90% of the regular or random noise generated by the CPAP device. In another embodiment, the method may include wherein the audio output device comprises one or more selected from the group consisting of (i) ear buds, (ii) headphones, (iii) a flexible audio output disk (e.g., for placement during use on a pillow or integrated into the pillow), and (iv) a flexible audio output material (e.g., integrated into a CPAP mask, headgear, or other patient circuit interface device).

According to yet another embodiment, the method may include wherein the patient interface device includes a CPAP headgear component, and wherein the audio output device is either (i) physically attached to the CPAP headgear component or (ii) physically separate from the CPAP headgear component. In another embodiment, the patient interface device comprises one selected from the group consisting of a nasal mask, a full facial mask, and a CPAP patient interface device configuration other than the nasal mask and full facial mask. For example, the CPAP patient interface device configuration may include a nasal pillow mask.

In yet another embodiment, the method may include wherein the microphone is coupled inside a housing of the base station of the CPAP device for receiving the regular or random noise generated by the CPAP device at least during the delivery of the pressurized flow of breathable gas. For example, the microphone is preferably located inside the base station housing, adjacent to or within a predetermined limited distance from, but not inside, the blower or blower housing which provides the flow of breathable gas. In other words, the predetermined distance can comprise a distance which maximizes a noise signal strength of the regular or random noise emanating from the blower and/or blower housing during delivery of the pressurized flow of breathable gas. Such a predetermined proximal distance is distinguishable from distal distances, e.g., in comparison to a location remote from the blower or blower housing within the base station housing which may contribute to a diminished noise signal strength.

The embodiments of the present disclosure advantageously solve or overcome the problem of CPAP device noise. More particularly, the embodiments of the present disclosure advantageously allow the use of ANC in an environment (e.g., when applied to the ear of a person or receiver as closely as possible) in which it works very well, instead of trying to make it work in an application (e.g., multi-point noise sources emanating into open 3D space) in which it does not work so well. According to the embodiments of the present disclosure, the overall application is suppression of CPAP device noise for patients and bed partners at night. One aspect of making the embodiments successful for their intended purpose is to provide a comfortable form that would not cause discomfort or sores in the ear or ear canal of a user and a form that will not affect the user's ability to sleep.

Still further advantages and benefits will become apparent to those of ordinary skill in the art upon reading and understanding the following detailed description.

### BRIEF DESCRIPTION OF THE DRAWINGS

The embodiments of the present disclosure may take form in various components and arrangements of components, and in various steps and arrangements of steps. Accordingly, the drawings are for purposes of illustrating the various embodiments and are not to be construed as limiting the embodiments. In the drawing figures, like reference numerals refer to like elements. In addition, it is to be noted that the figures may not be drawn to scale.

FIG. 1 is a block diagram view of a continuous positive airway pressure (CPAP) device featured with active noise cancellation (ANC) according to an embodiment of the present disclosure;

FIG. 2 is a block diagram view of the controller of the CPAP device featured with ANC of FIG. 1 in further detail according to an embodiment of the present disclosure;

FIG. 3 is a perspective view of a patient interface device of the CPAP device featured with ANC, in the form of a total face mask, according to an embodiment of the present disclosure;

FIG. 4 is a perspective view of a patient interface device of the CPAP device featured with ANC, in the form of a nasal mask, according to an embodiment of the present disclosure;

FIG. 5 is a perspective view of a patient interface device of the CPAP device featured with ANC, in the form of a nasal mask with an audio output device located within a pillow, according to an embodiment of the present disclosure;

FIG. 6 is a perspective view of a patient interface device of the CPAP device featured with ANC, in the form of a nasal mask with an attached patient circuit, according to another embodiment of the present disclosure;

FIG. 7 is a perspective view of a patient interface device of the CPAP device featured with ANC, in the form of a full face mask, according to an embodiment of the present disclosure; and

FIG. 8 is a flow diagram view of a method of active noise cancellation with a continuous positive airway pressure CPAP device according to an embodiment of the present disclosure.

### DETAILED DESCRIPTION

The embodiments of the present disclosure and the various features and advantageous details thereof are explained more fully with reference to the non-limiting examples that are described and/or illustrated in the drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the present disclosure. The examples used herein are intended merely to facilitate an understanding of ways in which the embodiments of the present may be practiced and to further enable those of skill in the art to practice the same. Accordingly, the examples herein should not be construed as limiting the scope of the embodiments of the present disclosure, which is defined solely by the appended claims and applicable law.

It is understood that the embodiments of the present disclosure are not limited to the particular methodology, protocols, devices, apparatus, materials, applications, etc., described herein, as these may vary. It is also to be understood that the terminology used herein is used for the purpose of describing particular embodiments only, and is not intended to be limiting in scope of the embodiments as claimed. It must be noted that as used herein and in the appended claims, the singular forms “a,” “an,” and “the” include plural reference unless the context clearly dictates otherwise.

Unless defined otherwise, all technical and scientific terms used herein have the same meanings as commonly understood by one of ordinary skill in the art to which the embodiments of the present disclosure belong. Preferred methods, devices, and materials are described, although any methods and materials similar or equivalent to those described herein can be used in the practice or testing of the embodiments.

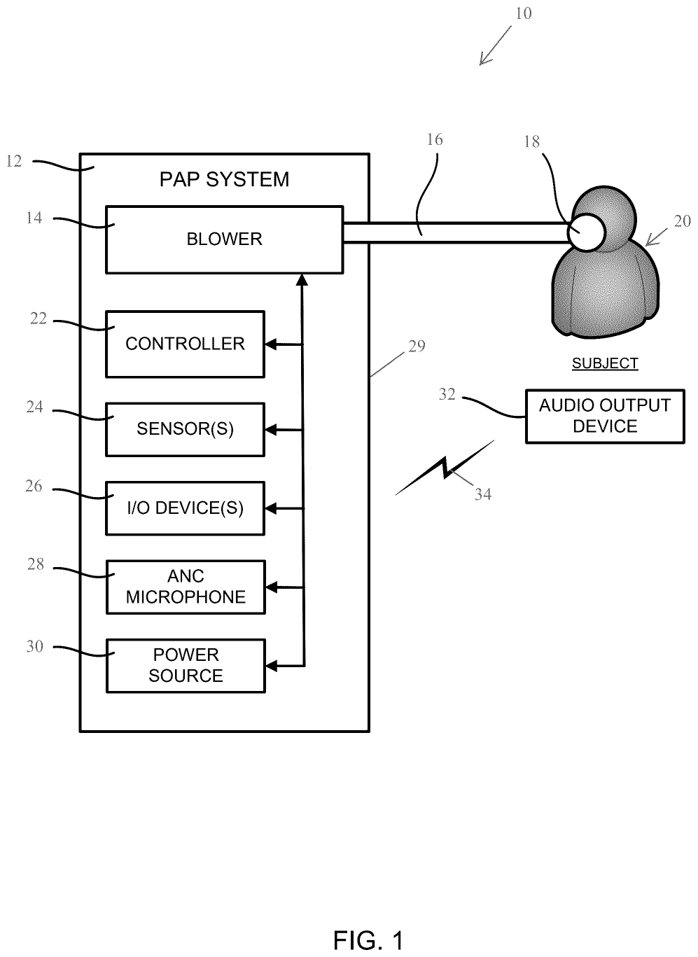

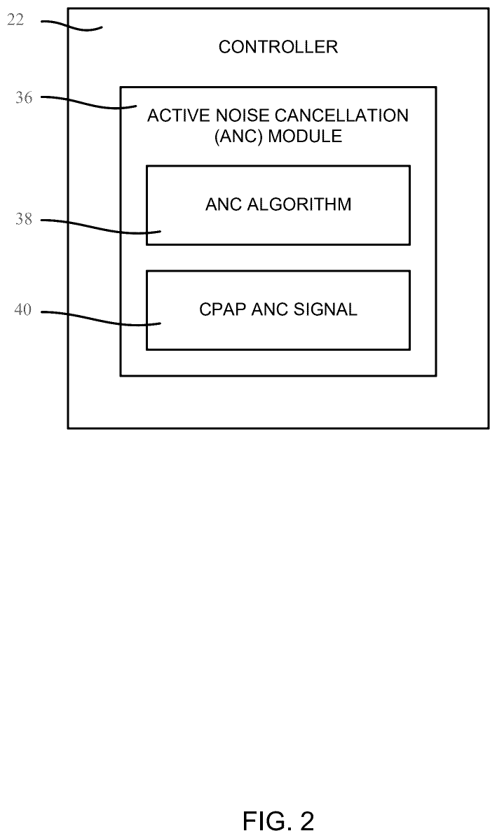

With reference to FIG. 1, there is shown a block diagram view of a continuous positive airway pressure (CPAP) system or device 10 featured with active noise cancellation (ANC) according to an embodiment of the present disclosure. The CPAP system 10 comprises a positive airway pressure device 12 having a gas flow generator or blower 14 configured to generate the flow of breathable gas. The gas flow generator 14 is configured to communicate the flow of breathable gas to a patient circuit 16 that includes a patient interface device or mask 18 to a patient or subject 20. The system further includes a controller 22 configured to implement one or more modules, as will be explained with reference to FIG. 2. In one embodiment, controller 22 comprises one or more of a microprocessor, microcontroller, field programmable gate array (FPGA), integrated circuit, discrete analog or digital circuit components, hardware, software, firmware, or any combination thereof, for performing various functions as discussed herein, further according to the requirements of a given PAP system implementation and/or application.

Controller 22 can further comprise one or more various modules configured to implement a given functionality as discussed herein. For example, the modules may comprise one or more of an integrated circuit, discrete analog or digital circuit components, hardware, software, firmware, or any combination thereof, for performing various functions as discussed herein, further according to the requirements of a given active noise cancellation (ANC) implementation and/or application according to an embodiment of the present disclosure. In addition, one or more of the modules may further comprise various combinations of one or more of the various modules as discussed herein. Furthermore, it is understood that the described modules may be computer program modules which are rendered in a non-transitory computer-readable medium.

One or more sensor 24 (or sensors) are provided and configured to generate output signals related to at least one characteristic associated with at least one of respiratory, actigraphy, airflow, humidity, pressure, temperature, in-mask sensor signals, or any combination thereof. First sensor signals can comprise respiratory signals, actigraphy signals, or a combination of both. Second sensor signals can comprise airflow sensor signals, pressure sensor signals, in-mask sensor signals, IR sensor signals (e.g., to detect a distance between a PAP mask and a user's forehead), or a combination thereof.

One or more input/output (I/O) device(s), collectively indicated by reference numeral 26, is representative of any number of suitable I/O devices for inputting or outputting one or more of an audible, visual, and/or tactile input or output, according to the requirements of a given PAP system implementation featured with active noise cancellation (ANC), as discussed herein. For example, input/output device(s) 26 can comprise one or more of an input/output device, a user interface, tactile output device, touch screen, optical display, microphone (e.g., for receiving user voice commands/responses), keypad, keyboard, pointing device, image capture device, video camera, audio output device, and any combination thereof, according to the requirements of the given PAP system implementation featuring active noise cancellation (ANC). In another embodiment, input/output device 26 may include a mobile phone app configured for inputting or outputting one or more of an audible, visual, and/or tactile input or output, according to the requirements of a given PAP system implementation featuring active noise cancellation (ANC).

With reference still to FIG. 1, the continuous positive airway pressure (CPAP) device 10 featured with active noise cancellation (ANC) further comprises a dedicated ANC microphone 28 located within base station housing 29, a power source 30, and at least one audio output device 32. Microphone 28 may comprise any suitable microphone or an accelerometer with a sufficiently wide band, as appropriate, for a given CPAP system implementation. In one embodiment, the dedicated ANC microphone 28 is preferably located inside the base station housing 29, adjacent to or within a predetermined limited distance from, but not inside, the blower 14 or blower housing. The predetermined limited distance can comprise a distance which maximizes a noise signal strength of the regular or random noise emanating from the blower and/or blower housing during delivery of the pressurized flow of breathable gas. Such a predetermined proximal distance is distinguishable from distal distances, e.g., in comparison to a location remote from the blower or blower housing within the base station housing which may contribute to a diminished noise signal strength. Also as shown in FIG. 1, the lightning bolt identified via reference numeral 34 is indicative of a wired or wireless link (e.g., near field communications, Bluetooth™ or other wireless communications link) between a given component (e.g., controller 22) of the PAP system 12 and the at least one audio output device 32.

Referring now to FIG. 2, there is shown a block diagram view of the controller 22 of the CPAP device 10 featured with ANC of FIG. 1 in further detail. In one embodiment, controller 22 comprises an active noise cancellation (ANC) module 36 configured to implement an active noise cancellation ANC algorithm for (i) processing the captured regular or random noise and (ii) generating an ANC output signal based on the processed regular or random noise. The active noise cancellation (ANC) module 36 may include an ANC algorithm module 38 and a CPAP ANC signal module 40.

The ANC algorithm module 38 is configured to process the captured regular or random noise via a suitable active noise cancellation algorithm. Active noise cancellation algorithms are generally known in the art. One example of an active noise cancelling algorithm is disclosed in U.S. Pat. No. 8,948,410, issued Feb. 3, 2015, entitled “Active Audio Noise Cancelling,” assigned to the assignee of the present disclosure, which is incorporated herein by reference in its entirety. Another example is disclosed in U.S. Pat. No. 9,165,549, issued Oct. 20, 2015, entitled “Audio Noise Cancelling,” assigned to the assignee of the present disclosure, which is incorporated herein by reference in its entirety. The active noise cancelling algorithm processes at least the regular or random noise captured via the ANC microphone 28 which is acoustically coupled to the CPAP device. More particularly, the dedicated ANC microphone 28 is acoustically coupled in a location within the base station housing 29 of the CPAP device proximate the blower or air flow generator 14. In this manner, the ANC microphone is adapted to principally capture the regular or random noise generated by or at the CPAP device at least during the delivery of the pressurized flow of breathable gas to the subject. In addition, the ANC microphone may also capture noise in the patient circuit.

The CPAP ANC signal module 40 is configured to provide the ANC output signal based on the regular or random noise of the CPAP device which has been processed via the ANC algorithm module 38. The ANC output signal is provided to the audio output device 32, wherein the audio output device produces a CPAP specific noise cancelling audio signal. Specifically, the CPAP specific noise cancellation signal seeks to provide a signal with an opposite phase of the regular or random noise (e.g., corresponding sound waves) arriving at the ANC microphone 28, thereby resulting in a destructive interference that at least partly cancels out the regular or random CPAP device noise in the audio environment of the CPAP device. The active noise cancellation (ANC) module 36 may also implement a feedback loop which generates the sound canceling signal based on the audio signal measured by the ANC microphone 28 in the presence of both the noise and the noise cancellation signal. As a result, a sound level of the regular or random noise generated by the CPAP device is advantageously reduced and/or eliminated at the subject during the delivery of the pressurized flow of breathable gas.

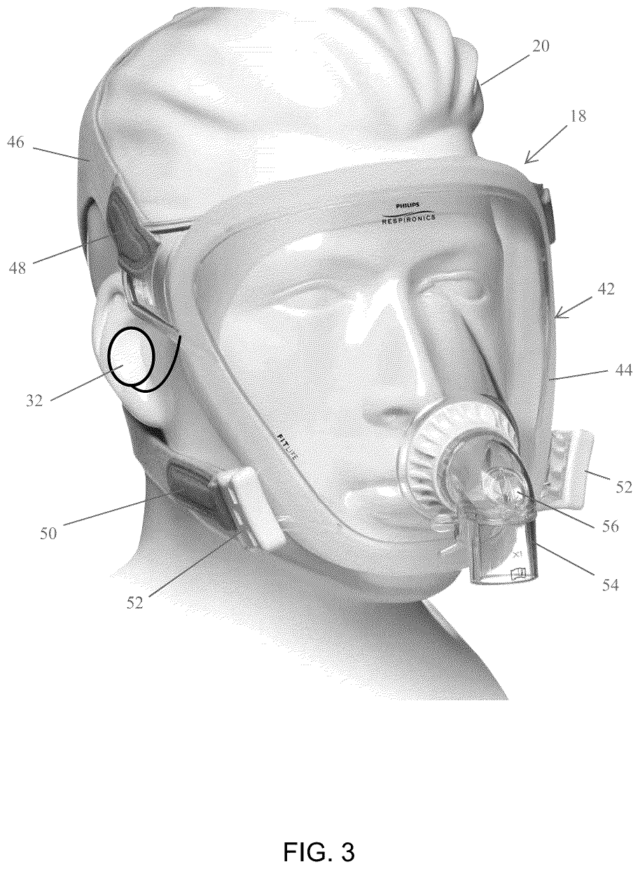

With reference now to FIG. 3, there is shown a perspective view of a patient interface device 18 of the CPAP device 10 featured with ANC, in the form of a total face mask 42, according to an embodiment of the present disclosure. As shown, the total face mask 42 doesn't contact the areas on and around the user's nose, but seals (e.g., via an over-molded cushion integrated into the mask frame 44 or faceplate) around the perimeter of the user's face. The total face mask 42 includes a large surface area to equalize pressure and flow inside the mask, minimizing eye irritation. The mask 42 further includes a headgear component 46 (e.g., one or more low-stretch headgear straps that provide stability and a consistent fit) that physically couples to the total face mask via headgear adjustment tabs 48,50 located at upper and lower left and right sides of the face mask. The headgear tabs can include large inlaid headgear tabs for quick, easy adjustment and removal of the headgear. The upper adjustment tabs 48 attach to respective left and right locations on the frame 44 of the total face mask. The lower adjustment tabs 50 attach to respective lower left and right sides of the face mask frame via snap clips 52. The snap clips 52 enable quick set-up and removal (e.g., subsequent to an initial fitting of the mask to the user's head) of the headgear strap 46 from the frame of the face mask 42 when a user desires to put on or take off the mask, respectively. The snap clips thus simplify set-up and eliminate the need to refit the mask after removal.

The total face mask 42 further includes a patient circuit elbow connector 54 equipped of an entrainment valve 56. The entrainment valve 56 allows access to room air if pressure drops below 3 cm H₂O. According to one embodiment, the at least one audio output device 32 is physically attached to the frame 44 of the total face mask 40 proximate the location of at least one of the user's ears. In one embodiment, the at least one audio output device 32 is an integral soft flexible component part of the mask frame 44. In another embodiment, the at least one audio output device 32 is coupled to the mask frame 44 via a suitable lanyard. The at least one audio output device can be configured as an inner ear piece or as an outer ear cover, or a combination of both.

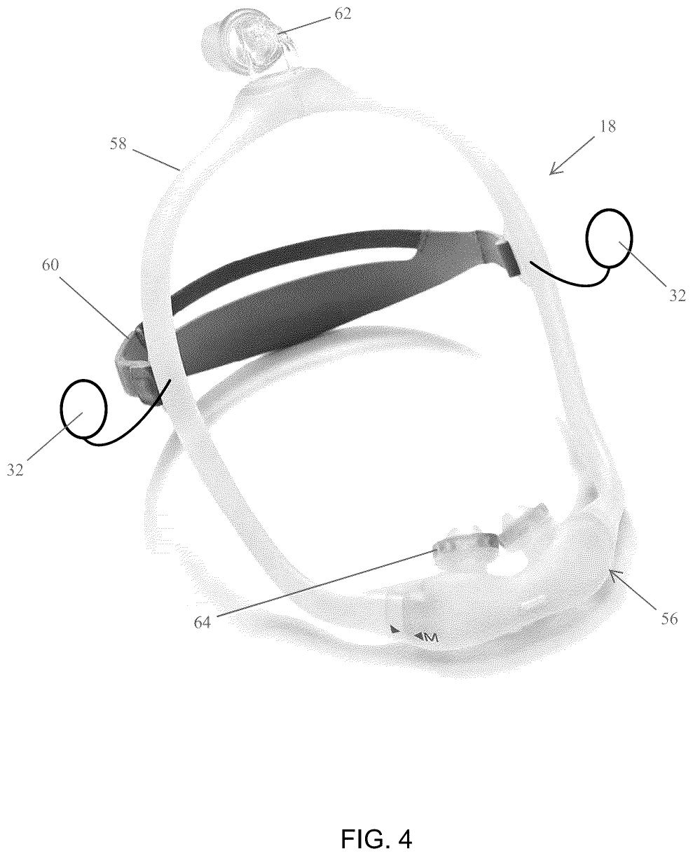

Turning now to FIG. 4, a perspective view is shown of a patient interface device of the CPAP device 10 featured with ANC, in the form of a nasal mask 56, according to an embodiment of the present disclosure. The nasal mask 56 is configured to direct airflow through the frame 58 (e.g., a soft silicone frame) so patients can wear glasses, watch TV and sleep comfortably. Mask 56 includes a headgear component 60 (e.g., one or more low-stretch headgear straps and/or arms of a soft, slip-resistant design that provide stability and a consistent fit) that physically couples to the nasal mask frame 58 via headgear adjustment tabs located at left and right sides of the frame. A patient circuit elbow connector 62 (i.e., tubing connection) is located at the top of the frame 58 (and thus on top of a user's head) which allows patients when wearing the mask to sleep in any position—stomach, side or back.

Still further, the nasal mask 56 comprises a silicone pillows cushion mask that conforms to various nose shapes and sizes. The nasal mask 56 is an under-the-nose design. It features a silicone pillows cushion 64 which is soft to the touch, softer than gel pillows, which deliver an exceptional nostril seal. According to one embodiment, the at least one audio output device 32 is physically attached to the frame 58 of the nasal mask 56 proximate the location of at least one of the user's ears. In one embodiment, the at least one audio output device 32 is an integral soft flexible component part of the mask frame 58. In another embodiment, the at least one audio output device 32 is coupled to the mask frame 58 via a suitable lanyard. The at least one audio output device can be configured as an inner ear piece or as an outer ear cover, or a combination of both.

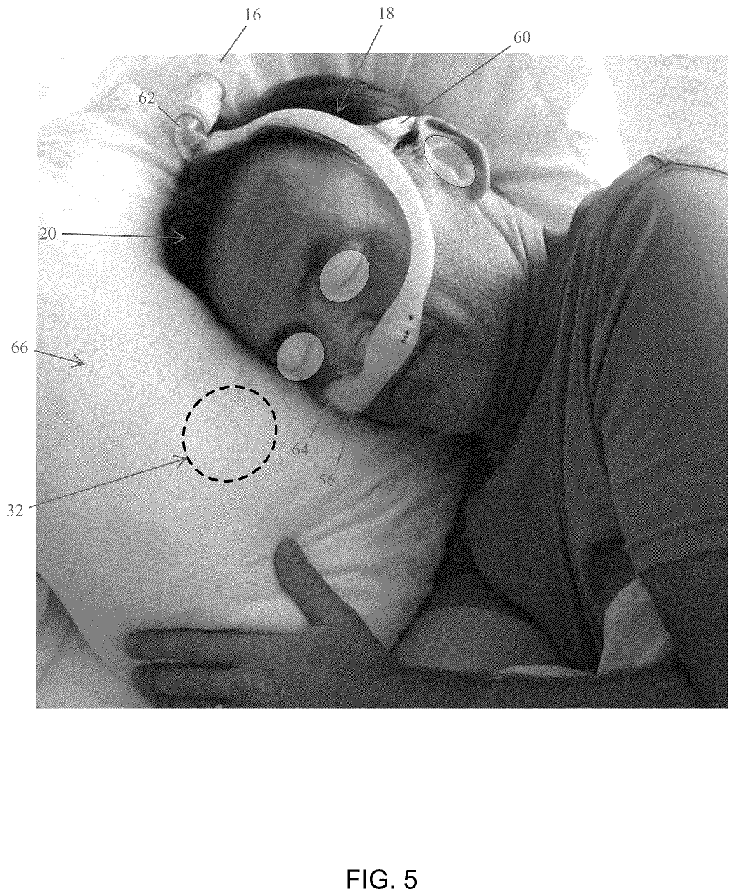

Turning now to FIG. 5, there is shown a perspective view of a patient interface device 18 of the CPAP device 10 featured with ANC, in the form of a nasal mask 56. The embodiment of FIG. 5 is similar to that of FIG. 4, with the exception that the audio output device 32 is located within a pillow 66, according to an embodiment of the present disclosure.

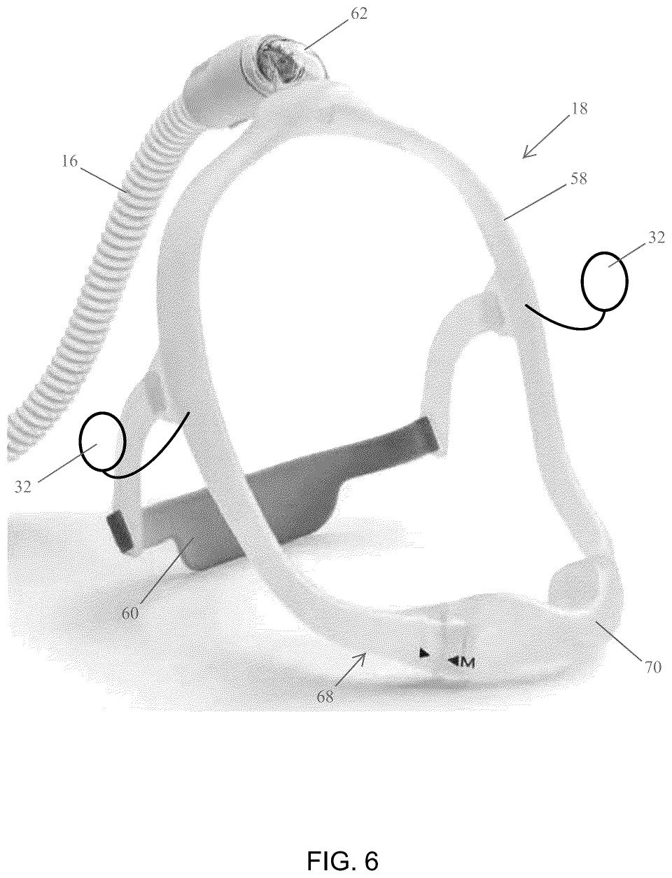

With reference now to FIG. 6, a perspective view is shown of a patient interface device 18 of the CPAP device 10 featured with ANC, in the form of a nasal mask 68 with an attached patient circuit 16, according to another embodiment of the present disclosure. The embodiment of FIG. 6 is similar to that of FIG. 4, with the exception of an under-the-nose nasal cushion 70 in place of the nasal gel pillow cushion 64, according to an embodiment of the present disclosure. In this embodiment, the cushion opening is placed directly under the user's nostrils, whereby the cushion 64 hugs the user's nose and leaks will be minimal.

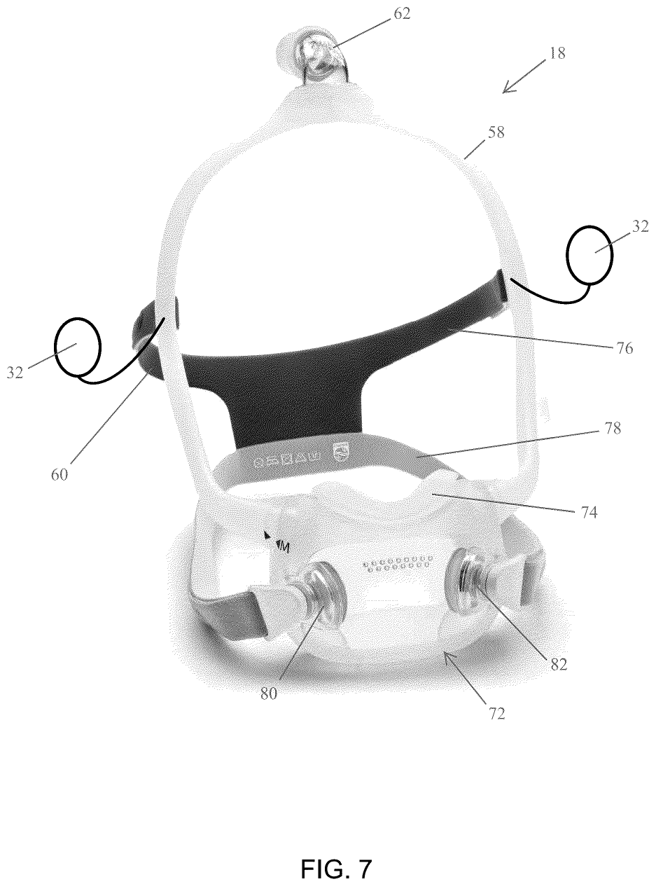

Turning now to FIG. 7, there is shown a perspective view of a patient interface device 18 of the CPAP device 10 featured with ANC, in the form of a full face mask 72, according to an embodiment of the present disclosure. The embodiment of FIG. 7 is similar to that of FIG. 4, with the exception of a cushion 74 in place of the nasal gel pillow cushion 64, according to an embodiment of the present disclosure. In this embodiment, the cushion opening is directly under the user's nostrils, whereby the cushion 74 hugs the user's nose and mouth, and leaks will be minimal. In addition, the headgear component 60 includes a top strap 76 coupled to a bottom strap 78. The top strap 76 physically couples to the frame 58 of the full face mask 72 via headgear adjustment tabs located at upper left and right sides of the mask. The lower strap 78 physically couples to the frame 58 of the full face mask 72 via headgear clips 80,82 located at lower left and right sides of the mask.

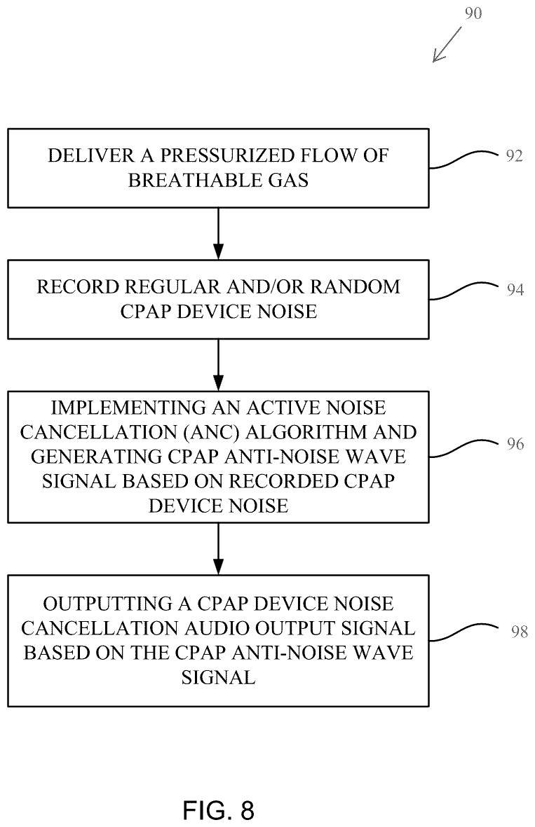

FIG. 8 is a flow diagram view of a method 90 of active noise cancellation with a continuous positive airway pressure CPAP device according to an embodiment of the present disclosure. A first step 92 includes delivering a pressurized flow of breathable gas to a subject, via a patient interface device fluidly coupled to a base station of the CPAP device via a patient circuit. During at least the delivery of the pressurized flow of breathable gas, the CPAP device produces regular and/or random CPAP device noise. For example, the regular CPAP device noise can consist of noise having a given duty cycle or periodic characteristic behaviour, whereas random CPAP device noise may consist of noise other than regular CPAP device noise. In one embodiment, the regular and/or random CPAP device noise may consist of more than 90% of noise which emanates from a blower alone in the base station of the CPAP device.

A second step 94 includes recording, via a microphone acoustically coupled to the CPAP device, the CPAP device noise produced by the CPAP device at least during the delivery of the pressurized flow of breathable gas to the subject. A third step 96 includes implementing, via a controller, an active noise cancellation (ANC) algorithm for (i) processing the recorded CPAP device noise and (ii) generating a CPAP anti-noise wave signal based on the processed CPAP device noise. A subsequent step 98 includes outputting, via an audio output device coupled to the patient interface device at a predetermined placement location of the patient interface device (e.g., proximate one or both ears of the subject) at least during the delivery of the pressurized flow of breathable gas, a CPAP device noise cancellation audio output signal based on the CPAP anti-noise wave signal. The CPAP device noise cancellation audio output signal is configured to reduce noise effects of the CPAP device by eliminating up to a given noise threshold amount of the regular and/or random noise produced by or at the CPAP device. The recording of CPAP noise may include time-based measurements over the course of a breath to capture not only the noise from the blower fan but also changes in fan noise due to the regular change in motor speed associated with compensation for circuit flow changes, circuit pressure drop and compensation, such as, tubing and mask compensation in the patient circuit.

According to one embodiment, the method may include wherein the predetermined placement location of the audio output device comprises a location of one or more component of the audio output device placed within one or both ears of a subject. According to another embodiment, the method may include wherein the predetermined placement location of the audio output device comprises a location of one or more component of the audio output device placed overlying both ears of a subject. According to yet another embodiment, the method may include wherein the placement location of the audio output device comprises a location of a component of the audio output device placed overlying at least one ear of a subject. According to a still further embodiment, the placement location of the audio output device may comprise a location of the audio output device placed within a given threshold distance from at least one ear of a subject.

According to another embodiment, the method includes wherein the given noise threshold amount can comprise at least one of (i) more than 50%, (ii) more than 75%, and (iii) more than 90% of the regular or random noise generated by the CPAP device. In another embodiment, the method may include wherein the audio output device comprises one or more selected from the group consisting of (i) ear buds, (ii) headphones, (iii) a flexible audio output disk (e.g., for placement during use on a pillow or integrated into the pillow), and (iv) a flexible audio output material (e.g., integrated into a CPAP mask, headgear, or other patient circuit interface device).

According to yet another embodiment, the method may include wherein the patient interface device includes a CPAP headgear component, and wherein the audio output device is either (i) physically attached to the CPAP headgear component or (ii) physically separate from the CPAP headgear component. In another embodiment, the patient interface device comprises one selected from the group consisting of a nasal mask, a full facial mask, and a CPAP patient interface device configuration other than the nasal mask and full facial mask.

In yet another embodiment, the method may include wherein the microphone is coupled inside a housing of the base station of the CPAP device for receiving the regular or random noise generated by the CPAP device at least during the delivery of the pressurized flow of breathable gas. For example, the microphone is preferably located inside the base station housing, adjacent to or within a predetermined limited distance from, but not inside, the blower or blower housing, which provides the flow of breathable gas. In other words, the predetermined distance can comprise a distance which maximizes a noise signal strength of the regular or random noise emanating from the blower and/or blower housing during delivery of the pressurized flow of breathable gas. Such a predetermined proximal distance is distinguishable from distal distances, e.g., in comparison to a location remote from the blower or blower housing within the base station housing which may contribute to a diminished noise signal strength.

The measures/device features which are disclosed herein to solve the identified problems, and provide resulting advantages, include one or more of the following: A pervasive design goal for continuous positive airway pressure (CPAP) devices is to make such devices quieter. The embodiments of the present disclosure make use of active noise cancellation (ANC) within a CPAP device as a means of reaching this goal. ANC does not work well when applied directly to multi-point noise sources emanating into open three-dimensional (3D) space. However, ANC does work well in small aperture systems like headphones and ear buds. According to the embodiments of the present disclosure, the CPAP device with active noise cancellation includes a type of ear covering for use with CPAP that advantageously addresses CPAP noise at the recipient, not at the source. The embodiments of the present disclosure are tailored for advantageously reducing noise effects of a CPAP or Bi-Level device.

Although only a few exemplary embodiments have been described in detail above, those skilled in the art will readily appreciate that many modifications are possible in the exemplary embodiments without materially departing from the novel teachings and advantages of the embodiments of the present disclosure. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

In addition, any reference signs placed in parentheses in one or more claims shall not be construed as limiting the claims. The word “comprising” and “comprises,” and the like, does not exclude the presence of elements or steps other than those listed in any claim or the specification as a whole. The singular reference of an element does not exclude the plural references of such elements and vice-versa. One or more of the embodiments may be implemented by means of hardware comprising several distinct elements, and/or by means of a suitably programmed computer. In a device claim enumerating several means, several of these means may be embodied by one and the same item of hardware. The mere fact that certain measures are recited in mutually different dependent claims does not indicate that a combination of these measures cannot be used to an advantage.

1. A continuous positive airway pressure CPAP device featured with active noise cancellation, comprising:
a patient interface device configured for being fluidly coupled to a base station of the CPAP device via a patient circuit for delivery of a pressurized flow of breathable gas from the CPAP device to the patient interface device;
a microphone adapted for being acoustically coupled to the CPAP device for capturing regular or random noise generated by or at the CPAP device at least during the delivery of the pressurized flow of breathable gas;
a controller adapted to implement an active noise cancellation ANC algorithm for (i) processing the captured regular or random noise and (ii) generating an ANC output signal based on the processed regular or random noise; and
an audio output device adapted for being coupled to the patient interface device at a predetermined placement location of the patient interface device at least during the delivery of the pressurized flow of breathable gas, wherein the audio output device receives the ANC output signal and generates, in response to receipt of the ANC output signal, a CPAP device noise cancellation audio output configured to reduce noise effects of the CPAP device by eliminating up to a given noise threshold amount of the noise generated by or at the CPAP device.

2. The device according to claim 1, wherein the predetermined placement location of the audio output device comprises a location of one or more component of the audio output device placed within one or both ears of a subject.

3. The device according to claim 1, wherein the predetermined placement location of the audio output device comprises a location of one or more component of the audio output device placed overlying both ears of a subject.

4. The device according to claim 1, wherein the placement location of the audio output device comprises a location of a component of the audio output device placed overlying at least one ear of a subject.

5. The device according to claim 1, wherein the placement location of the audio output device comprises a location of the audio output device placed within a given threshold distance from at least one ear of a subject.

6. The device according to claim 1, wherein the given noise threshold amount comprises at least one of (i) more than 50%, (ii) more than 75%, and (iii) more than 90% of the regular or random noise generated by the CPAP device.

7. The device according to claim 1, wherein the audio output device comprises one or more selected from the group consisting of (i) ear buds, (ii) headphones, (iii) a flexible audio output disk, and (iv) a flexible audio output material.

8. The device according to claim 1, wherein the patient interface device includes a CPAP headgear component, and wherein the audio output device is either (i) physically attached to the CPAP headgear component or (ii) physically separate from the CPAP headgear component.

9. The device according to claim 1, wherein the patient interface device comprises one selected from the group consisting of a nasal mask, a full facial mask, and a CPAP patient interface device configuration other than the nasal mask and full facial mask.

10. The device according to claim 1, wherein the microphone is coupled inside a housing of the base station of the CPAP device, adjacent to or within a predetermined limited distance from, but not inside, a blower or blower housing, for receiving the regular or random noise generated by the CPAP device at least during the delivery of the pressurized flow of breathable gas.

11. A method of active noise cancellation with a continuous positive airway pressure CPAP device, comprising:
delivering a pressurized flow of breathable gas, via a patient interface device fluidly coupled to a base station of the CPAP device via a patient circuit, wherein at least during the delivery of the pressurized flow of breathable gas the CPAP device produces regular or random CPAP device noise;
recording, via a microphone acoustically coupled to the CPAP device, the CPAP device noise produced by the CPAP device at least during the delivery of the pressurized flow of breathable gas;
implementing, via a controller, an active noise cancellation (ANC) algorithm for (i) processing the recorded CPAP device noise and (ii) generating a CPAP anti-noise wave signal based on the processed CPAP device noise; and
outputting, via an audio output device coupled to the patient interface device at a predetermined placement location of the patient interface device at least during the delivery of the pressurized flow of breathable gas, a CPAP device noise cancellation audio output signal based on the CPAP anti-noise wave signal, the CPAP device noise cancellation audio output signal configured to reduce noise effects of the CPAP device by eliminating up to a given noise threshold amount of the noise produced by or at the CPAP device.

12. The method according to claim 11, wherein the predetermined placement location of the audio output device comprises a location of one or more component of the audio output device placed within one or both ears of a subject.

13. The method according to claim 11, wherein the predetermined placement location of the audio output device comprises a location of one or more component of the audio output device placed overlying both ears of a subject.

14. The method according to claim 11, wherein the placement location of the audio output device comprises a location of a component of the audio output device placed overlying at least one ear of a subject.

15. The method according to claim 11, wherein the placement location of the audio output device comprises a location of the audio output device placed within a given threshold distance from at least one ear of a subject.

16. The method according to claim 11, wherein the given noise threshold amount comprises at least one of (i) more than 50%, (ii) more than 75%, and (iii) more than 90% of the regular or random noise produced by or at the CPAP device.

17. The method according to claim 11, wherein the audio output device comprises one or more selected from the group consisting of (i) ear buds, (ii) headphones, (iii) a flexible audio output disk, and (iv) a flexible audio output material.

18. The method according to claim 11, wherein the patient interface device comprises a CPAP headgear component, and wherein the audio output device comprises is either (i) physically attached to the CPAP headgear component or (ii) physically separate from the CPAP headgear component.

19. The method according to claim 11, wherein the patient interface device comprises one selected from the group consisting of a nasal mask, a full facial mask, and a CPAP patient interface device configuration other than the nasal mask and full facial mask.

20. The method according to claim 11, wherein the microphone is coupled inside a housing of the base station of the CPAP device, adjacent to or within a predetermined distance from, but not inside, a blower or blower housing, for receiving the regular or random noise generated by the CPAP device at least during the delivery of the pressurized flow of breathable gas.
